(19) United States
(12) Patent Application Publication
(10) Pub. No.: US 2011/0226801 A1
(43) Pub. Date: Sep. 22, 2011

(54) DISPENSER AND ASSOCIATED METHODS

(76) Inventor: Christy Smith-Heskel, Malabar, FL (US)

(21) Appl. No.: 13/050,582

(22) Filed: Mar. 17, 2011

Related U.S. Application Data
(60) Provisional application No. 61/315,601, filed on Mar. 19, 2010.

(51) Int. Cl. B67D 7/84

(52) U.S. Cl. 222/1; 222/173

(57) ABSTRACT

A dispenser includes a pair of opposing side bracket members, a top member, a push button member, and a pair of applicator members. Each of the pair of opposing side bracket members may include an outer surface portion and an inner surface portion. The inner surface portion of each of the opposing side bracket members may include a pair of applicator member receiving recess and a first sanitizer container receiving recess. The top member may overlie and may be removeably connected to the pair of opposing side bracket members when the opposing side bracket members are connected to one another. The top member may include an outer surface portion and an inner surface portion. The inner surface portion may include a second sanitizer container receiving recess. One of the respective pair of opposing applicator members may be carried by one of the applicator member receiving recesses when the pair of opposing side bracket members are connected to one another.

### RELATED APPLICATIONS

This application claims the benefit of U.S. Provisional Patent Application Ser. No. 61/315,601 filed on Mar. 19, 2010 by the inventor of the present application, titled “Antimicrobial Agent Dispenser and Associated Methods,” the entire contents of which are incorporated herein by reference.

### FIELD OF THE INVENTION

The present invention relates to the field of surface sanitization and, more specifically, to the field of devices that dispense an antimicrobial agent onto a substrate, along with associated methods of use.

### BACKGROUND OF THE INVENTION

People touch many surfaces with their hands as they go about their daily routines. Often, the surfaces people touch have previously been exposed to bacteria, viruses or other illness-causing contaminants, which have been deposited thereon by, for example, a previous user. If these surfaces are not sanitized, bacterial or viral infections may be passed to users who contact such surfaces. Certain other illnesses may also be transferred from one person to another in this manner. As such, there is a need for devices and methods to quickly and easily remove bacteria, viruses and other illness-causing contaminants from surfaces before touching them.

The handle of a shopping cart, which may, for example, be used at a grocery store or other retail store, is one example of a surface that can often have many illness-causing contaminants deposited thereon. A shopper may touch freshly butchered meat items, for example, contaminating the shopper's hands with animal blood and bacteria, and then may touch the handle of a shopping cart. Also, produce items may have harsh chemical pesticides that can also become deposited on a shopping cart handle, after a shopper picks up such items and then touches the cart handle. A shopper who is ill may use such a shopping cart and may transmit to a subsequent shopper the germs that they are carrying by contaminating the shopping cart handle.

Still other shoppers may be carriers of methicillin-resistant staphylococcus aureus (MRSA), and may transmit MRSA to the shopping cart handle. MRSA can be a deadly virus that can be easily transmitted from a MRSA carrier. Another shopper, who may, for example, have an open wound or a weakened immune system, may come in contact with a shopping cart handle contaminated with MRSA, and may contract a MRSA infection from that contact, which may result in death when untreated. The reality is that, after a single use by a shopper, the handle of a shopping cart may be significantly contaminated, presenting a health danger for each subsequent user.

Current solutions to this problem involve providing sanitary wipes that a user may employ to remove contaminants from a surface. This solution can be inadequate when the surface to be sanitized is capable of being moved, as is the case with a shopping cart handle, because sanitary wipes are typically dispensed from a single stationary location, which may be distant from where a user desires to sanitize a surface. Also, sanitary wipe dispensers must be frequently refilled with sanitary wipes and often are found empty by a user. Additionally, a user may not properly dispose of a sanitary wipe after use, creating litter and another potential method of spreading illness-causing contaminants.

Another proposed solution has been to provide sterilized plastic that can be positioned between a user's hands and a contaminated surface, as illustrated, for example, in U.S. Pat. Nos. 5,429,377 and 5,820,142, both to Duer. Again, this solution involves providing some kind of dispenser that must be refilled and potential litter from improper disposal of used plastic. Additionally, sterilized plastics may be slow to degrade in landfills, adding to the amount of un-reusable waste that modern society produces. The use of plastic coverings can also be difficult for persons with low dexterity caused by, for example, having small children in tow.

Other proposed solutions, which are designed to remove illness-causing materials from handling surfaces, involve permanently attaching both a sleeve and a sanitizer to encircle a handling surface, as in U.S. Pat. No. 7,611,156 to Dunser. However, these solutions are inferior in that the permanently attached members may not be removed for easy cleaning and refilling. Additionally, such solutions must be affixed to a handling surface during manufacture, and as such are not readily suited to retrofitting of existing handling surfaces.

Accordingly, a solution is necessary to address the problem of infection being spread between users of devices, such as shopping carts that have handles.

### BRIEF SUMMARY OF THE INVENTION

With the above in mind, it is therefore an object of the present invention to provide a dispenser that can be readily attached to a handle of, for example, a shopping cart. The present invention advantageously provides a dispenser that can be readily detached from the handle of a shopping cart for simple cleaning. The present invention further advantageously provides a dispenser that reduces waste that may be introduced into a landfill. The present invention still further advantageously provides a dispenser that can be readily used by multiple users without significantly reduced danger of transmitting disease and illness. The present invention also advantageously provides a dispenser that can be readily used to clean a surface, and positioned out of a user's way when using the handle of the shopping cart.

These and other objects, features and advantages in accordance with the present invention are provided by a dispenser comprising a pair of opposing side bracket members, a top member, a push button member, and a pair of applicator members. Each of the pair of opposing side bracket members may include an outer surface portion and an inner surface portion. The inner surface portion of each of the opposing side bracket members may include a pair of applicator member receiving recess and a first sanitizer container receiving recess. The member may overlie and may be removeably connected to the pair of opposing side bracket members when the opposing side bracket members are connected to one another. The top member may include an outer surface portion and an inner surface portion. The inner surface portion may include a second sanitizer container receiving recess. One of the respective pair of opposing applicator members may be carried by one of the applicator member receiving recesses when the pair of opposing side bracket members are connected to one another.

The first sanitizer container receiving recess may be formed adjacent a top portion of each of the side bracket members. Each of the first sanitizer container receiving recesses may have a shape substantially similar to a quarter arc. Each of the applicator member receiving recesses may have a substantially semi-circular shape. Each of the applicator member receiving recesses may be aligned with one another to form a single applicator member receiving recess that has a substantially circular shape when the side bracket members are connected to one another. Each of the pair of first sanitizer container receiving recesses may be aligned with one another to form a single sanitizer container receiving recess that has a substantially semi-circular shape when the side bracket members are connected to one another.

The pair of first sanitizer container receiving recesses and the second sanitizer container receiving recess forms a single sanitizer container receiving recess having a substantially circular shape when the top member overlies and is removeably connected to the side bracket members that are connected to one another. Each of the side bracket members may include at least one side fastener receiving passageway formed therein to receive a respective at least one fastener to connect the side bracket members to one another. Each of the side bracket members may include at least one top fastener receiving passageway formed therein to receive a respective at least one, top fastener to connect the top member to the side bracket members when the side bracket members are connected to one another.

The push button member may be moveable between an engaged position and a disengaged position. More particularly, the push button member may matingly engages a portion of the sanitizer container receiving recess formed when the top member connected to the side bracket members that are connected to one another.

A method aspect of the present invention is for using the dispenser. The method may comprise removeably connecting the pair of applicator members to a handle. The method may also include positioning the pair of opposing side bracket members to engage the pair of applicator members connected to the handle so that applicator member receiving recesses formed on an inner surface portion of each of the side bracket members surround the pair of applicator members. The method my further include connecting each of the side bracket members to one another by passing at least one fastener through at least one fastener receiving passageway formed through each of the side bracket members. The method may still further include positioning a sanitizer container on a first sanitizer container receiving recess formed on the inner surface portion of the side bracket members.

The method may also include positioning the top member to overlie the pair of opposing side bracket members that are connected to one another so that the top member overlies the sanitizer container on the first sanitizer container receiving passageway and so that a second sanitizer container receiving passageway engages the sanitizer container. The method may further include connecting the top member to each of the side bracket members by passing at least one fastener through at least one fastener receiving passageway formed in the top member and into a respective at least one top fastener receiving passageway formed in each of the side bracket members.

### BRIEF DESCRIPTION OF THE DRAWINGS

FIG. 1 is a perspective view of a dispenser according to an embodiment of the present invention.

FIG. 2 is an exploded perspective view of the antimicrobial agent dispenser illustrated in FIG. 1.

FIG. 3 is a perspective view of a top member of the dispenser illustrated in FIG. 1.

FIG. 4 is a perspective view of one of the side bracket members of the dispenser illustrated in FIG. 1.

FIG. 5 is a perspective view of one of the side bracket members of the dispenser illustrated in FIG. 1.

FIG. 6 is a perspective view of a push-button member of the dispenser illustrated in FIG. 1.

FIG. 7 is a perspective view of a pair of applicator members of the dispenser illustrated in FIG. 1.

### DETAILED DESCRIPTION OF THE INVENTION

The present invention will now be described fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

The present invention is directed to a dispenser 100. The dispenser 100 is preferably an antimicrobial agent dispenser, which is suitable for attaching to a shopping cart handle. Those of ordinary skill in the art will realize that the following description of the embodiments of the present invention are only illustrative and are not intended to be limiting in any way. Other embodiments of the present invention will readily suggeSt themselves to such skilled persons having the benefit of this disclosure.

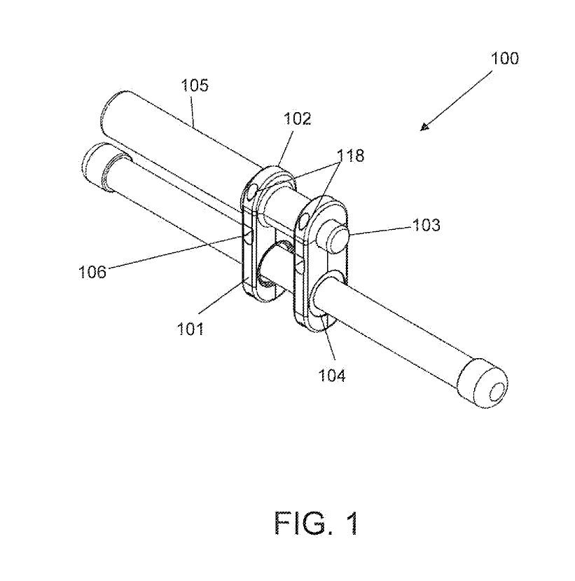

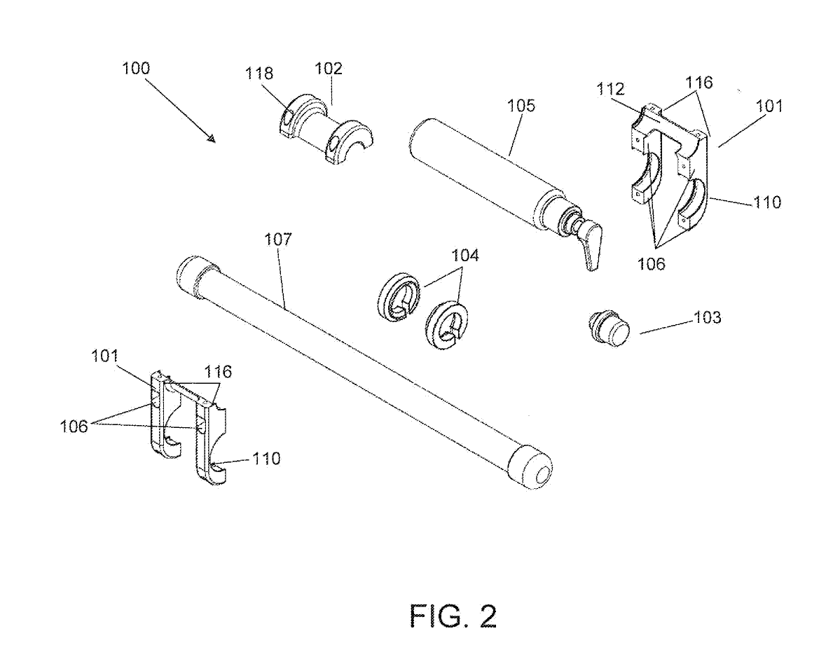

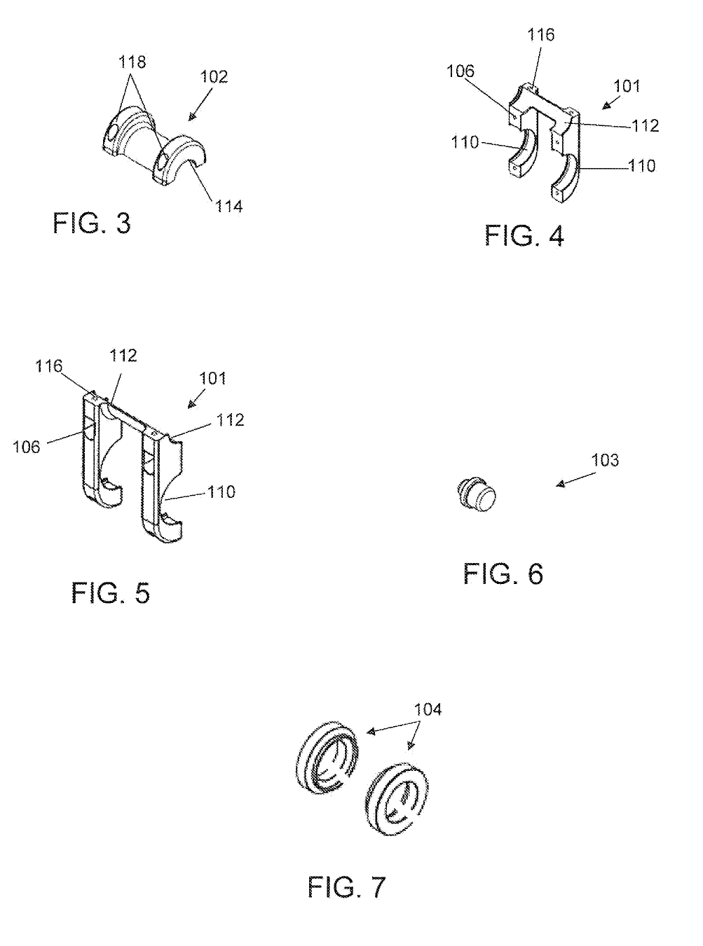

FIG. 1 illustrates a perspective view of the dispenser 100, according to an embodiment of the present invention. FIG. 2 illustrates an exploded view of the dispenser 100. The dispenser 100, according to an embodiment of the present invention, may include a pair of side bracket members 101 that illustratively oppose one another. The dispenser 100 also includes a top member 102, a push button member 103, and a pair of applicator members 104. When assembled, as illustrated in FIG. 1, the dispenser 100 is adapted to hold a sanitizer container 105. The sanitizer container 105 is preferably provided by a sanitizer bottle, as illustrated, and may, for example, be a standard sized 8 ounce bottle of sanitizer. Those skilled in the art will appreciate, however, that any size container may be used to contain the sanitizer.

It is preferable that the sanitizer container 105 be of the type that includes a top mounted, pump style, dispenser that dispenses a sanitizing gel. Accordingly, and as will be discussed in greater detail below, the sanitizing gel may be dispensed from the sanitizer container 105 onto a shopping cart handle 107 by depressing the pump by engaging the push button member 103 of the dispenser 100. Thereafter, a user can laterally move the dispense 100 laterally along the shopping cart handle 107 to spread the sanitizer that has been dispensed thereon. The dispenser 100 according to the present invention advantageously allows for the user to apply an antimicrobial sanitizing agent to the shopping cart handle 107 and spread the antimicrobial agent along portions of the shopping cart handle so as to reduce the risk of spreading disease through contact. Additional details of use of the dispense 100 according to embodiments of the present invention will be provided below.

After viewing FIG. 2, it will become apparent to a person skilled in the art that the size and shape of the side bracket members 101 and the top bracket member 102 are chosen such that, when the bracket pieces are joined, a structure is formed, which may carry the sanitizer container 105 in a position parallel to the shopping cart handle. The push button member 103 may be in a position adjacent the sanitizer container 105 and, more particularly, adjacent the top mounted pump dispensing portion of the sanitizer container 105 so that engagement of the push button member will cause dispensing of the sanitizer carried by the sanitizer container. Further, the sanitizer is preferably dispensed onto the handle 107 adjacent the applicator members 104 in a position adjacent the handle.

In a preferred embodiment, the pair of side bracket members 101 may have, two vertically oriented members connected to each other by a horizontally oriented member at a top end thereof. The horizontally oriented member of each side bracket member 101 may have an inner surface shaped to support the sanitizer container 105. The inner surface shaped to support the sanitizer container 105 may define a first sanitizer container receiving recess 112. In other words, each of the pair of opposing side bracket members 101 may include an outer surface portion and an inner surface portion. The inner surface portion of each of the side bracket members 101 may include the first sanitizer container receiving recess 112. The first sanitizer container receiving recesses 112 may be formed adjacent a top portion of each of the side bracket members 101. Further, each of the first sanitizer container receiving recesses 112 may illustratively have a shape substantially similar to a quarter arc. Accordingly, when the pair of side bracket members 101 are connected to one another, the first sanitizer container member receiving recess 110 may illustratively have a semi-circular shape.

After having had the benefit of reading this disclosure, those skilled in the art will appreciate that the side bracket members 101, while depicted in FIGS. 1-2 as having two vertically oriented members, may have any number of vertically oriented members, each of which may be connected to the next by a horizontally oriented member, while still accomplishing the goals, features and objectives according to the present invention. Furthermore, those skilled in the art will appreciate that the side bracket members 101, while depicted in FIGS. 1-2 as having a horizontally oriented member with a semicircular-shaped inner surface, may have a horizontally oriented member with a square-shaped, triangular-shaped, rectangular-shaped, trapezoidal-shaped or any other-shaped inner surface in order to support a portion of an appropriately shaped sanitizer container 105 or an appropriately shaped button member 103.

As indicated above, the vertical members of each side bracket 101 may have an inner surface shaped to accept the pair of applicator members 104. More specifically, the inner surface portion of each of the opposing side bracket members 101 may include a pair of applicator member receiving recesses 110. Each of the applicator member receiving recesses 110 may illustratively have a semi-circular shape. After having had the benefit of reading this disclosure, those skilled in the art will appreciate that each of the applicator member receiving recesses 110 formed in each of the side bracket members 101, may have a square shape, a triangle shape, a rectangle shape, a trapezoid shape, or any other shape that is suitable to accept several different shaped applicator members 104. Accordingly, the dispenser 100 according to an embodiment of the present invention may advantageously be used to accommodate a handle 107 having any type of shape. More particularly, some retail outlets may have a desire to set themselves apart from their competitors by providing shopping carts having handles with various shapes. The dispenser 100 according to the present invention can advantageously be readily used in connection with any type of handle 107 on any type of shopping cart.

It is preferably that the applicator members 104 are shaped to substantially surround the handle 107. The applicator members 104 may have a gap formed through an outer peripheral portion thereof, and may be formed of a pliable type of material to be capable of engaging different diameter shopping cart handles. As perhaps best illustrated in FIG. 7, the gap may be formed in an outer peripheral portion of each of the applicator members 104 so that portions of each of the applicator members adjacent, the gap can be spaced apart and removeably connected to the handle 107 during the installation process. Similarly, the gap formed in each of the applicator members 104 advantageously allows each of the applicator members to be readily removed from the handle 107 when changing of the applicator members becomes necessary. After having had the benefit of reading this disclosure, those skilled in the art will appreciate that the applicator members 104, while depicted as having a circular shape, may have a square shape, a triangular shape, a rectangular shape, a trapezoidal shape, or any other shape in order to substantially surround a handle 107 upon which it is desired to dispense sanitizer (also referred to herein as an antimicrobial agent) thereon.

When the side bracket members 101 are connected to one another (as illustrated in FIG. 1) the pair of applicator member receiving recesses 110 on each of the side bracket members may be aligned with one another to form a single applicator receiving recess, i.e., when the side bracket members are joined with one another, two single applicator members receiving recesses are formed (one forward and one rear) that are adapted to surround the applicator members 104 and, by extension, the handle 107 to which the applicator members are connected. The single applicator member receiving recesses 110 that are formed when the side bracket members 101 are connected to one another have a substantially circular shape. As indicated above, however, the substantially circular shape of each of the applicator member receiving recesses 110 illustrated in the appended drawings are not meant to be limiting. Instead, those skilled in the art will appreciate that the applicator member receiving recesses 110 may have any desired shape that is suitable for the handle 107 to which it is to be mated. The applicator member receiving recesses 110 are shaped so that the applicator members 104 may be readily carried by thereby when the pair of side bracket members 101 are connected to one another.

The top member 102 may be positioned to overlie and may be removeably connected to the pair of opposing side bracket members 101 when the side bracket members are connected to one another. For example, and as perhaps best illustrated in FIGS. 1 and 2, when each of the side bracket members 101 are connected to one another so that the applicator member receiving recesses 110 surround the applicator members 104, the top member 102 may be positioned over the side bracket members and fastened thereto. The top member may have and outer surface and an inner surface, and the inner surface may have a second sanitizer container receiving recess 114. The sanitizer container 105 may be held in place by the first sanitizer container receiving recesses 110 formed on each of the side bracket members 101 and the second sanitizer container receiving recess 114 formed on the top member 102.

Each of the side bracket members 101 may have at least side fastener receiving passageway 106 formed therein. The at least side fastener receiving passageway 106 may be used to secure the pair of side bracket members 101 to each other. More specifically, each of the side fastener receiving passageway 106 may receive a respective at least one fastener to connect the side bracket members 101 to one another. As illustrated in FIGS. 4 and 5, it is preferably that the side fastener receiving passageways 106 are formed so that a fastener may be passed through one of the side fastener receiving passageways (for example, through the side fastener receiving passageway formed through the side bracket member illustrated in FIG. 5) and be received by another side fattener receiving passageway (for example, received by the side fastener receiving passageway formed in the side bracket member illustrated in FIG. 4). In the illustrated embodiment, it is preferably that a threaded fastening member be passed through the side fastener receiving passageways formed in the side bracket member 101 illustrated in FIG. C, and be received by a female threaded side fastening receiver 106 formed in the side bracket member illustrated in FIG. 4. Those skilled in the art will appreciate, however, that this configuration is for illustrative purposes only, and that any other type of configuration may be used to fasten the side bracket members 101 to one another. Further, those skilled in the art will appreciate that, although the illustrated embodiment of the dispenser 100 calls for using four fasteners to connect the side bracket members 101 to one another, any number of fasteners and any corresponding number of side fastener member receiving passageways, may be used to connect the side bracket members to one another.

A top portion of each of the side bracket members 101 may also include a top fastener receiving passageway 116 formed therein for receiving a fastener to connect the top member 102 to the side bracket members when the side bracket members are connected to one another. Similarly, the top member 102 may have at least one fastener receiving passageway 118 formed therethrough. The fastener receiving passageway 118 formed in the top member may receive a fastener that can be passed therethrough and that may be received by the top fastener receiving passageways 116 formed in the side bracket members 101 to secure the top member to the side bracket members. Again, the fastener may be a threaded fastener that may be passed through the fastener receiving passageway 118, and the top fastener receiving passageways 116 formed in the side bracket members 101 may be threaded to receive the fastener. Although the dispenser 100 illustrated in the appended drawings show that four fasteners can be used to secure the top member 102 to the side bracket members 101 when the side bracket members are connected to one another, those skilled in the art will appreciate that any number of fasteners may be used to secure the top member to the side bracket members.

The top member 102 may be shaped to overlie a portion of the sanitizer container 105 and the push button member 103. After having had the benefit of reading this disclosure, those skilled in the art will appreciate that the top member 102, while depicted in FIGS. 1-2 as having a semi-cylindrical shape, may have a square shape, a triangular shape, a rectangular shape, a trapezoidal shape, or any other-shape in order to overlie an appropriately shaped portion of the sanitizer container 105 and the button 103. The push button member 103 is moveable between an engaged position and a disengaged position. More specifically, and as perhaps best illustrated in FIG. 1, the push button member 103 matingly engages portions of the first and second sanitizer container receiving recesses 112, 114 formed in top portion of the side bracket members 101 and in the top member 102 when the top member is connected to the side bracket members that are, in turn, connected to one another. Accordingly, moving the push button member 103 between the engaged position and the disengaged position allows for sanitizer in the sanitizer container 105 to be readily dispensed onto the handle 107 so that the sanitizer can thereafter be spread on the handle by moving the dispense 100 back and forth to spread the sanitizer using the applicator members 104. Additional details of use of the dispenser 100 according to the present invention will be described in greater detail below.

The junction of the top member 102 with the pair of side bracket members 101 forms a pair of openings opposite each other. The sanitizer 105 may be supported in one such opening. The push button member 103 may be supported in the other such opening. The push button member 103 may have an upper end and a lower end. The sanitizer container 105 may have a pump nozzle or a spray nozzle for dispensing the antimicrobial agent (or sanitizer). When the sanitizer container 105 is supported in one opening formed by the junction of the top member 102 with the side bracket members 101, and the push button member 103 is supported in the other such opening, the lower end of the push button member may be positioned adjacent the nozzle of the sanitizer container.

A non-limiting example of a method of using the dispenser 100 according to an embodiment of the present invention to sanitize, for example, the handle 107 of a shopping cart is now described. A user may engage the pair of applicator members 104 with the shopping cart handle 107. After engaging the applicator members 104 with the shopping cart handle, the user may engage a side bracket member 101 with the shopping cart handle by engaging each side of a shopping cart handle so that an inner surface portion of each side bracket member 101 is aligned to matingly engage the applicator members 104 adjacent the applicator member receiving recesses 110. The user may secure the side bracket members 101 to one another by passing fasteners through the side fastener receiving passageways 106 formed in the side bracket members. After securing the side bracket members 101 to one another, the user may position the sanitizer container 105 on top of the joined side bracket members so that the nozzle of the sanitizer container is located between the vertical members of the side bracket members and is oriented toward the surface of the shopping cart handle 107, and so that the sanitizer container 105 is supported on the first sanitizer container receiving recesses 112.

The user may also place the push button member 103 on top of the joined side bracket members 101 so that the lower end of the push button member is positioned adjacent the nozzle of the sanitizer container 105. After placing the sanitizer container 105 and the push button member 103 on the joined side bracket members 101, the user may place the top member 102 on the joined side bracket members. The user may secure the top member 102 to the side bracket members 101 by passing fasteners through the fastener receiving passageways 118 formed through the top bracket 102 and into the top fastener receiving passageways 116 formed in each of the side bracket members 101. The user may then push the upper end of the push button member 103 to move the push button member from the disengaged position to the engaged position in order to dispense the sanitizer from the sanitizer container 105 onto the shopping cart handle 107 between the applicators 104. The user may thereafter move the dispenser 100 back and forth across the length of the shopping cart handle 107 in order to place the shopping cart handle in contact with the antimicrobial agent. More specifically, moving the dispenser 100 laterally over the shopping cart handle 107 allows the applicator members 104 to be used to spread the sanitizer that has been dispensed onto the handle and sanitize the handle prior to using.

One advantage of this configuration of the dispenser 100 according to the present invention is that when the applicator members 104 engage the shopping cart handle 107, the connection between the applicator members and the shopping cart handle are such that the dispenser may, if desired be readily rotated with respect to the shopping cart handle. Accordingly, when use of the dispenser 100 is required, the user may simply rotate the dispenser about the shopping cart handle 107, dispense a predetermined amount of sanitizer from the sanitizer container 105, move the dispenser back and forth so that the applicators cause the sanitizer to be spread over the shopping cart handle, and allow the dispenser to be rotated back to a position beneath the shopping cart handle. This allows for the dispenser 100 to be out of the way of the user when the user is using the shopping cart. Yet another advantage of the configuration of the dispenser 100 according to the present invention is that a user, if so desired, may also dispense some of the sanitizer onto their own hands to also sanitize their hands as well as the shopping cart handle 107.

The dispenser 100 may be provided with a locking mechanism so that the side bracket members 101 may be securely maintained to the shopping cart handle 107. It is preferable for all of the dispensers 100 associated, for example, with one set of shopping cart handles 107, i.e., associated the shopping carts of one particular retail outlet, to have a similar locking mechanism that can be readily opened using a single unlocking mechanism. For example, the locking mechanism may be provided using a key lock, and the single unlocking mechanism may be provided by a master key maintained by store management. Similarly, the locking mechanism may be a lock that may be opened by a combination, and the unlocking mechanism may be a single combination maintained by store management.

Many modifications and other embodiments of the invention will come to the mind of one skilled in the art having the benefit of the teachings presented in the foregoing descriptions and the associated drawings. Therefore, it is understood that the invention is hot to be limited to the specific embodiments disclosed.

That which is claimed is:

1. A dispenser comprising:
a pair of opposing side bracket members;
a top member;
a push button member; and
a pair of applicator members;
wherein each of the pair of opposing side bracket members includes an outer surface portion and an inner surface portion, and wherein the inner surface portion of each of the opposing side bracket members includes a pair of applicator member receiving recess and a first sanitizer container receiving recess;
wherein the top member overlies and is removeably connected to the pair of opposing side bracket members when the opposing side bracket members are connected to one another and wherein the top member includes an outer surface portion and an inner surface portion, the inner surface portion including a second sanitizer container receiving recess; and
wherein one of the respective pair of opposing applicator members are carried by one of the applicator member receiving recesses when the pair of opposing side bracket members are connected to one another.

2. A dispenser according to claim 1 wherein the first sanitizer container receiving recess is formed adjacent a top portion of each of the side bracket members.

3. A dispenser according to claim 2 wherein each of the first sanitizer container receiving recesses has a shape substantially similar to a quarter arc.

4. A dispenser according to claim 3 wherein each of the applicator member receiving recesses has a substantially semi-circular shape.

5. A dispenser according to claim 4 wherein each of the applicator member receiving recesses are aligned with one another to form a single applicator member receiving recess that has a substantially circular shape when the side bracket members are connected to one another.

6. A dispenser according to claim 4 each of the pair of first sanitizer container receiving recesses are aligned with one another to form a sanitizer container receiving recess that has a substantially semi-circular shape when the side bracket members are connected to one another.

7. A dispenser according to claim 6 wherein the pair of first sanitizer container receiving recesses and the second sanitizer container receiving recess form a sanitizer container receiving recess having a substantially circular shape when the top member overlies and is removeably connected to the side bracket members that are connected to one another.

8. A dispenser according to claim 1 wherein each of the side bracket members includes at least one side fastener receiving passageway formed therein to receive a respective at least one fastener to connect the side bracket members to one another.

9. A dispenser according to claim 1 wherein each of the side bracket members includes at least one top fastener receiving passageway formed therein to receive a respective at least one top fastener to connect the top member to the side bracket members when the side bracket members are connected to one another.

10. A dispenser according to claim 1 wherein the push button member is moveable between an engaged position and a disengaged position, and matingly engages a portion of the sanitizer container receiving recess formed when the top member connected to the side bracket members that are connected to one another.

11. A dispenser comprising:
a pair of opposing side bracket members including at least one side fastener receiving passageway formed therein to receive a respective at least one fastener to connect the pair of opposing side bracket members to one another and at least one top fastener receiving passageway formed therein to receive a respective at least one top fastener;
a top member including at least one receiving passageway formed therein;
a push button member; and
a pair of applicator members;
wherein each of the pair of opposing side bracket members includes an outer surface portion and an inner surface portion, and wherein the inner surface portion of each of the opposing side bracket members includes a pair of applicator member receiving recess each having a substantially semi-circular shape, and a first sanitizer container receiving recess that is formed adjacent a top portion of each of the side bracket members and that has a shape substantially similar to a quarter arc;
wherein the top member overlies and is removeably connected to the pair of opposing side bracket members when the opposing side bracket members are connected to one another and wherein the top member includes an outer surface portion and an inner surface portion, the inner surface portion including a second sanitizer container receiving recess;
wherein one of the respective pair of opposing applicator members are carried by one of the applicator member receiving recesses when the pair of opposing side bracket members are connected to one another;
wherein the first sanitizer container receiving recess is formed adjacent a top portion of each of the side brackets;
wherein the at least one top fastener receiving passageway formed in a top portion of each of the side bracket members is adapted to receive a respective at least one fastener to connect the top member to the side bracket members when the side bracket members are connected to one another;
wherein the at least one fastener receiving passageway formed in the top member is adapted to receive at least one fastener to connect the top member to the side bracket members when the side bracket members are connected to one another; and
wherein the push button member is moveable between an engaged position and a disengaged position, and matingly engages a portion of the sanitizer container receiving recess formed when the top member connected to the side bracket members that are connected to one another.

12. A dispenser according to claim 11 wherein each of the applicator member receiving recesses are aligned with one another to form a single applicator member receiving recess that has a substantially circular shape when the side bracket members are connected to one another.

13. A dispenser according to claim 11 each of the pair of first sanitizer container receiving recesses are aligned with one another to form a sanitizer container receiving recess that has a substantially semi-circular shape when the side bracket members are connected to one another.

14. A dispenser according to claim 11 wherein the pair of first sanitizer container receiving recesses and the second sanitizer container receiving recess form a sanitizer container receiving recess having a substantially circular shape when the top member overlies and is removeably connected to the side bracket members that are connected to one another.

15. A method of using a dispenser that includes a pair of opposing side bracket members, a top member, a push button member, and a pair of applicator members, the method comprising:
removeably connecting the pair of applicator members to a handle, each of the pair of applicator members having a substantially circular shape and a gap formed through a portion thereof so that portions of each of the applicator members adjacent the gap can be spaced apart to connect to the handle;
positioning the pair of opposing side bracket members to engage the pair of applicator members connected to the handle so that applicator member receiving recesses formed on an inner surface portion of each of the side bracket members surround the pair of applicator members;
connecting each of the side bracket members to one another by passing at least one fastener through at least one fastener receiving passageway formed through each of the side bracket members;
positioning a sanitizer container on a first sanitizer container receiving recess formed on the inner surface portion of the side bracket members;
positioning the top member to overlie the pair of opposing side bracket members that are connected to one another so that the top member overlies the sanitizer container on the first sanitizer container receiving passageway and so that a second sanitizer container receiving passageway engages the sanitizer container; and
connecting the top member to each of the side bracket members by passing at least one fastener through at least one fastener receiving passageway formed in the top member and into a respective at least one top fastener receiving passageway formed in each of the side bracket members.

16. A method according to claim 15 further comprising moving the push button member between an engaged position and a disengaged position to dispense contents of the sanitizer container onto the handle.

17. A method according to claim 16 further comprising slidably moving the dispenser laterally along the handle to spread the contents of the sanitizer container that have been dispensed onto the handle.

18. A method according to claim 15 further comprising removing the top member from the side bracket members to replace the sanitizer container.
